(19) United States
(12) Patent Application Publication
(10) Pub. No.: US 2006/0269985 A1
(43) Pub. Date: Nov. 30, 2006

(54) Method for constructing array blocks, and tissue punching instrument and tissue blocks used therefor

(75) Inventor: Yasuhiko Kitayama, Shizuoka-shi (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignees: Yasuhiko Kitayama, Shizuoka-shi (JP); Kabushiki Kaisha Azumaya Ika Kikai, Tokyo (JP); Nihon Automatic Control Kabushiki Kaisha, Tokyo (JP)

(21) Appl. No.: 11/139,534

(22) Filed: May 31, 2005

(51) Int. Cl. C12M 1/36; G01N 1/30

(52) U.S. Cl. 435/40.52; 435/286.2

(57) ABSTRACT

A formalin fixed paraffin block an organ is fixed on a stage, the stage is moved and a front end of a tubular blade is relatively positioned at a target part, the tubular blade punches the tissue, the tissue is extracted by a tissue extracting rod, a tissue block has been prepared in advance having a plurality of holes having inner diameter very close to the outer diameter of the tubular blade, the extracted tissues are planted in the holes on an embedding pan so that the front end of the tissue is protrusively facing toward the embedding pan, and liquid paraffin is filled into a gap between the embedding pan and the tissue block and solidified, whereby the tissue and paraffin are integrated, and good array block sections are constructed easily.

### BACKGROUND OF THE INVENTION

1. Field of the Invention

The present invention relates to a method for constructing array blocks, and a tissue punching instrument and tissue blocks used therefor, applicable to general pathological diagnosis and research purposes.

2. Description of the Related Art

Recently, there has been provided an instrument named “Tissue Arrayer” from a company named “Beecher Instruments”, for constructing tissue arrays. The Tissue Arrayer has any of two hollow punchers among those having diameters of 2 mm, 1 mm and 0.6 mm, respectively. One of the two hollow punchers punches and obtains a tissue from a known paraffin block, and the other hollow puncher forms a hole in another paraffin block in which any tissue has not been planted. Thereafter, the obtained tissue paraffin pillar tissue is planted in the other paraffin block, in which, before such a planting, any tissue has not been planted. The position of obtained paraffin block would be determined by referring to hematoxylin-eosin (HE) stain samples. According to this prior art method, because the obtained sample is small, it is necessary to prepare a special slide glass, on which an adhesive tape has been attached. Further, the adhesive tape must be detached in a solvent, and the paraffin blocks must be overlapped. Thus, this type of tissue punching machine according to the prior art requires very complicated operation and long time, and in addition, because the tissue block has not been heated, when the tissue block is punched, there are many cases in which the tissue blocks are damaged. Further, due to the small tissue size, it is not suitable for ordinary pathological diagnosis, and they have been used chiefly for genetic research purposes.

According to the prior art as discussed above, the instrument for constructing tissue arrays, is comprising, a punch platform carriage movable in Z axis direction, a punch platform mounted on the punch platform carriage and displaceable between at least first and second positions with respect to the punch platform carriage so that such positions may precisely defined by detentes or stops, at least first and second punch units mounted on the punch platform so that each punch unit may comprise a punch and a cooperating stylet, means for holding the recipient block, means for selectively repositioning the recipient block and punch platform in X and Y axis with respect to each other, and means for guiding the movement of at least one of the recipient block and the punch platform carriage in Z axis relative to each other. The first punch unit comprises a recipient punch and associated stylet, and the second punch unit comprises a donor punch and associated stylet, so that the donor punch has an inner diameter greater than the recipient punch. When the punch platform is in the first position, the recipient punch is in position over the recipient block holder and in alignment with Z axis. On the other hand, when the punch platform is in the second position, the donor punch is in position over the recipient block holder and in alignment with Z axis. Accordingly, there are several merits, such as that the positioning of plural number of punches can be done continuously and accurately, that the positioning of punches can also be done by manual operation easily, that the automatic or semi-automatic construction of tissue arrays can be done, and that the two stylet punches can be alternatively positioned easily, from one punch to the other, with regard to tissue array constructing instrument (cf: Japanese National Patent Publication No. 2002-537794; PCT/US00/05824).

However, it is almost impossible, for the instrument and method for constructing tissue arrays according to the prior art as discussed above, to be applied to ordinary tissue diagnosis. For the purpose of special staining and immunostaining, and in situ hybridization, due to very small size of each tissue, it is difficult to carry out judgment and assessment accurately. Further, it is also difficult to find the difference of expressions of various proteins, etc. at various localizations. The positioning of obtained tissue is difficult, and there are many cases in which the tissue has been obtained at inaccurate position. The non-heating treatment of used block is possibly leading to high risk of breaking of the block. Because of planting in the solid paraffin block, the planted tissue and the paraffin block around the tissue will not be fit in easily, and there are several cases in which the good section cannot be obtained. Further, it is almost impossible to punch the tissue out of the fixed organ itself. The operation is also complicated, requiring much time, and the smooth operation would require the skill to some degree. An ordinary slide glass cannot be used, and instead, it is necessary to use a special slide glass, an adhesive tape and UV irradiation. Further, the production cost is high, and it may be said that there is almost no usage for ordinary pathology diagnosis. In the above situation, it is very critical problem to provide array blocks, which can be prepared by simple and easy method, suitable for ordinary pathological diagnosis.

### SUMMARY OF THE INVENTION

It is an object of the present invention to provide a method for constructing array blocks, and tissue punching instrument and tissue blocks used for that method, in which good tissue sections may be prepared easily.

To achieve the object mentioned above, according to claim 1 of the present invention, there is provided a method for constructing array blocks, comprising steps of: fixing a formalin fixed paraffin block of an organ on a stage; moving the stage and relatively positioning a front end of a tubular blade at a target part of the organ; punching tissue of the organ by the tubular blade; extracting the tissue from the tubular blade by a tissue extracting rod; preparing a tissue block in advance having a plurality of holes, each of the holes having inner diameter very close to the outer diameter of the tubular blade; planting extracted tissues in the holes of the tissue block placed on an embedding pan, so that the front end of the tissue is protrusively facing toward the embedding pan; and filling liquid paraffin into a gap between the embedding pan and the tissue block, and solidifying the liquid paraffin, whereby the tissue and paraffin are integrated.

According to claim 2 of the present invention, there is provided a method for constructing array blocks, comprising steps of: fixing an organ on a stage after formalin fixing; moving the stage and relatively positioning a front end of a tubular blade at a target part of the organ; punching tissue of the organ by the tubular blade; extracting the tissue from the tubular blade by a tissue extracting rod; preparing a tissue block in advance having a plurality of holes, each of the holes having inner diameter very close to the outer diameter of the tubular blade; planting extracted tissues in the holes of the tissue block fixed on an embedding pan, so that the front end of the tissue is protrusively facing toward the embedding pan; and filling liquid paraffin into a gap between the embedding pan and the tissue block, and solidifying the liquid paraffin, whereby the tissue and paraffin are integrated.

According to claim 3 of the present invention, there is provided the method for constructing array blocks as claimed in claim 1, characterized in that the formalin fixed paraffin block is heated at temperature from 40° C. to 50° C. by a heater of the stage.

According to claims 4 and 5 of the present invention, there is provided the method for constructing array blocks as claimed in claim 1 or claim 2, further characterized in that the liquid paraffin, filled into the gap between the embedding pan and the tissue block, is heated by a heater, whereby the tissue and paraffin are integrated.

According to claims 6 through 10 of the present invention, there is provided the method for constructing array blocks as claimed in any one claim among claims 1 to 5, further characterized in that the outer diameter of the tubular blade is from 3 mm to 7 mm.

According to claims 11 through 20 of the present invention, there is provided the method for constructing array blocks as claimed in any one claim among claims 1 through 10, further characterized in that vacuum suction is done inside the tubular blade during punching of tissue by the tubular blade.

According to claim 21 of the present invention, there is provided a tissue punching instrument comprising: a base; a tubular blade mounted on the base for punching tissue by an up-down operation mechanism; and a stage mounted on the base, for fixing a formalin fixed paraffin block, and for relatively positioning a front end of the tubular blade substantially at a target part of an organ through movement of the stage in X axis and Y axis directions by a horizontal positioning mechanism.

According to claim 22 of the present invention, there is provided the tissue punching instrument as claimed in claim 21, further comprising a heater for heating the formalin fixed paraffin block.

According to claims 23 and 24 of the present invention, there is provided the tissue punching instrument as claimed in claim 21 or claim 22, further characterized in that the outer diameter of the tubular blade is from 3 mm to 7 mm.

According to claims 25 through 28 of the present invention, there is provided the tissue punching instrument as claimed in any one claim among claims 21 through 24, further comprising a positioning pin provided on the base, selectively used in place of the tubular blade, for determining an accurate position of tissue at the target part.

According to claims 29 through 36, there is provided the tissue punching instrument as claimed in any one claim among claims 21 through 28, further comprising a vacuum suction apparatus inside the tubular blade.

According to claim 37, there is provided a tissue block, being able to be arranged on an embedding pan, characterized in that a plurality of holes has been formed, each of which inner diameter very close to the outer diameter of the tubular blade of the tissue punching instrument according to any one claim among claims 21 through 36.

Therefore, according to claim 1 or claim 2 of the present invention, the target tissue part of the formalin fixed paraffin block of the organ or the organ itself, which has been obtained by surgical operation or biological test, is punched, and the punched tissues are then planted in the holes of the tissue block fixed on the embedding pan, and the liquid paraffin is filled in the gap between the embedding pan and the tissue block. Thus, the tissue and the paraffin are integrated, and neat and stable array block can be obtained, whereby the good sections may be constructed easily.

With reference to the method for constructing the array blocks as discussed above, when the formalin fixed paraffin block is heated by the heater of the stage at temperature from 40° C. to 50° C., the paraffin block may be softened, and it is possible to punch the tissues without causing any damage or crack. Thus, neat and complete condition of tissues may be obtained.

Further, with reference to the method for constructing the array blocks as discussed above, when the liquid paraffin, which has been filled in a gap between the embedding pan and the tissue block arranged on the stage, is heated by the heater, the obtained tissues and the paraffin may be integrated smoothly.

Further, with reference to the method for constructing the array blocks as discussed above, when the outer diameter of the tubular blade is from 3 mm to 7 mm, it is possible to obtain relatively wider scope of tissues, sufficiently suitable for ordinary tissue diagnosis, special staining, immunostaining, fluorescence in situ hybridization (FISH method), and other research purposes.

Still further, with reference to the method for constructing the array blocks as discussed above, when the vacuum suction is done inside the tubular blade during punching of the tissue by the tubular blade, the punched tissue may be extracted without fail.

According to the tissue punching instrument as discussed above, it is possible to punch the tissues substantially at the target part easily.

The tissue punching instrument according to the present invention may be provided with the heater for heating the formalin fixed paraffin block fixed on the stage, whereby the paraffin block may be softened, and it is possible to punch the tissues without causing any damage or crack. Thus, neat and complete condition of tissues may be obtained.

The outer diameter of the tubular blade of the tissue punching instrument according to the present invention, may be from 3 mm to 7 mm. Therefore, it is possible to obtain relatively wider scope of tissues, sufficiently suitable for ordinary tissue diagnosis, special staining, immunostaining, fluorescence in situ hybridization (FISH method), and other research purposes.

The tissue punching instrument according to the present invention may be provided with the positioning pin for accurately positioning the tubular blade at the target tissue part. Therefore, it is possible to determine the target tissue part accurately, whereby the tissue punching operation may become very simplified, and even a fresh operator may handle the instrument easily within a short period of time.

Further, with reference to the tissue punching instrument according to the present invention, the tubular blade may be provided with the vacuum suction apparatus, whereby, during punching of the tissue, the punched tissue may be extracted without fail.

According to the tissue block of the present invention, the punched tissues are arranged by the holes, and may be planted in the holes so that each front end of the tissue is protrusively facing toward the embedding pan. When the liquid paraffin is filled in the gap between this tissue block and the embedding pan, and solidified, it is possible to construct the array block, in which the arranged tissues and the paraffin have been integrated, easily and efficiently.

Therefore, according to the method for constructing array blocks as claimed in claim 1, the tissues, which have been obtained by punching the target part of the organ tissue out of the formalin fixed paraffin block of the organ, are planted in the holes of the tissue block fixed on the embedding pan, and the array block is then obtained by wholly filling the liquid paraffin in the gap between the embedding pan and the tissue block. When the tissues and the paraffin are integrated and sufficiently solidified, the formed array block is removed from the embedding pan, whereby the tissues and the paraffin may be integrated sufficiently, and neat and stable array block may be constructed.

According to the method for constructing array blocks as claimed in claim 2, the organ tissues have been obtained by directly punching the formalin fixed organ. The punched organ tissues are then dehydrated and defatted by tissue embedding apparatus, and planted in the holes of the tissue block fixed on the embedding pan, and the array block is then obtained by wholly filling the liquid paraffin in the gap between the embedding pan and the tissue block. When the tissues and the paraffin are integrated and sufficiently solidified, the formed array block is removed from the embedding pan, whereby the tissues and the paraffin may be integrated sufficiently, and neat and stable array block may be constructed.

According to the method for constructing array blocks as claimed in claim 3, the formalin fixed paraffin block is heated at temperature from 40° C. to 50° C. by the heater of the stage. Therefore, the paraffin block may be softened, and it is possible to punch the tissues without causing any damage or crack. Thus, neat and complete condition of tissues may be obtained.

According to the method for constructing array blocks as claimed in claims 4 and 5, the liquid paraffin, filled into the gap between the embedding pan and the tissue block, is heated by the heater. Therefore, after the sample has been punched by the tubular blade and inserted in the tissue block, it is possible to integrate the obtained tissues and the paraffin, whereby neat and stable array block may be constructed.

According to the method for constructing array blocks as claimed in claims 6 through 10, the outer diameter of the tubular blade is from 3 mm to 7 mm. Therefore, it is possible to obtain relatively wider scope of tissues, sufficiently suitable for ordinary tissue diagnosis, special staining, immunostaining, fluorescence in situ hybridization (FISH method), and other research purposes.

According to the method for constructing array blocks as claimed in claims 11 through 20, the vacuum suction is done inside the tubular blade during punching of tissue by the tubular blade, whereby the tissue can be extracted without fail.

According to the tissue punching instrument as claimed in claim 21, it is possible to punch the tissues substantially at the target part easily.

According to the tissue punching instrument as claimed in claim 22, the heater for heating the formalin fixed paraffin block is provided. Therefore, the paraffin block may be softened, and it is possible to punch the tissues without causing any damage or crack. Thus, neat and complete condition of tissues may be obtained.

According to the tissue punching instrument as claimed in claims 23 and 24, the outer diameter of the tubular blade is from 3 mm to 7 mm. Therefore, it is possible to obtain relatively wider scope of tissues, sufficiently suitable for ordinary tissue diagnosis, special staining, immunostaining, fluorescence in situ hybridization (FISH method), and other research purposes.

According to the tissue punching instrument as claimed in claims 25 through 28, the positioning pin is provided for positioning the tubular blade at an accurate position of tissue at the target part. Therefore, it is possible to carry out the positioning of the target tissue accurately, whereby the tissue punching operation may also become very simplified, and even a fresh operator may handle the instrument easily within a short period of time. Thus, the total cost for operation may be reduced, and applicable scope may extend to ordinary pathological purposes.

According to the tissue punching instrument as claimed in claims 29 through 36, the vacuum suction apparatus is provided inside the tubular blade. Therefore, during punching of the tissue, the tissue can be extracted without fail.

Further, according to the tissue block as claimed in claim 37, it is possible to construct the array block easily and efficiently, in which the arranged tissues and the paraffin have been integrated.

### BRIEF DESCRIPTION OF THE DRAWINGS

The invention will be described below in detail with reference to the accompanying drawings, in which:

FIG. 1 is a front view of a tissue punching instrument according to a first embodiment of the present invention;

FIG. 2 is a right side view of the tissue punching instrument according to the first embodiment of the present invention;

FIG. 3 is a left side view of the tissue punching instrument according to the first embodiment of the present invention;

FIG. 4 is a perspective view of a horizontal direction positioning mechanism according to the first embodiment of the present invention;

FIG. 5 shows a front view of a positioning pin and partial section views of tubular blades;

FIG. 6 is a perspective view of a formalin fixed paraffin block on a stage;

FIG. 7 shows partial section views of the tubular blades by which tissues have been punched;

FIG. 8 shows perspective views in which the tissues are extracted from the tubular blades;

FIG. 9 is a front view of tissue blocks;

FIG. 10 is a perspective view in which the tissue blocks are mounted on an embedding pan;

FIG. 11 is a section view in which the tissues are inserted in holes of the tissue block;

FIG. 12 is a section view in which liquid paraffin is filled in a space between the embedding pan and the tissue block;

FIG. 13 is a front view of the completed tissue blocks;

FIG. 14 is a perspective view of the allay block and sliced sections;

FIG. 15 is a flow chart showing a method for constructing array blocks;

FIG. 16 is a perspective view showing relation of the embedding pan, the tissue block and an upper cover;

FIG. 17 is a perspective view in which the tissue block and the upper cover have been mounted on the embedding pan;

FIG. 18 is a section view in which the liquid paraffin has been filled in the space between the embedding pan and the tissue block and also in the upper cover;

FIG. 19 is a right side view of a tissue punching instrument according to a second embodiment of the present invention;

FIG. 20 is an expanded section view of a highlighted part of the tissue punching instrument;

FIG. 21 is a partial section view of the tubular blade;

FIG. 22 is a front view of the tissue extracting rod;

FIG. 23 is a front view of the tissue block;

FIG. 24 is a bottom view of the tissue block;

FIG. 25 is a partial section view of the tubular blade;

FIG. 26 is a front view of the tissue extracting rod;

FIG. 27 is a front view of the tissue block; and

FIG. 28 is a bottom view of the tissue block.

### DETAILED DESCRIPTION OF THE INVENTION

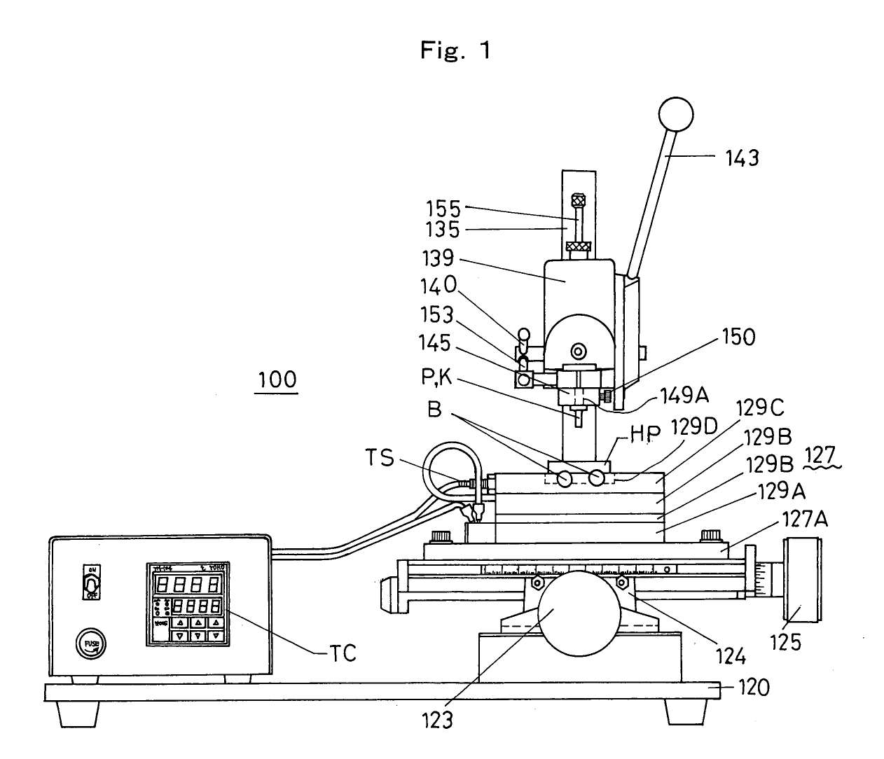

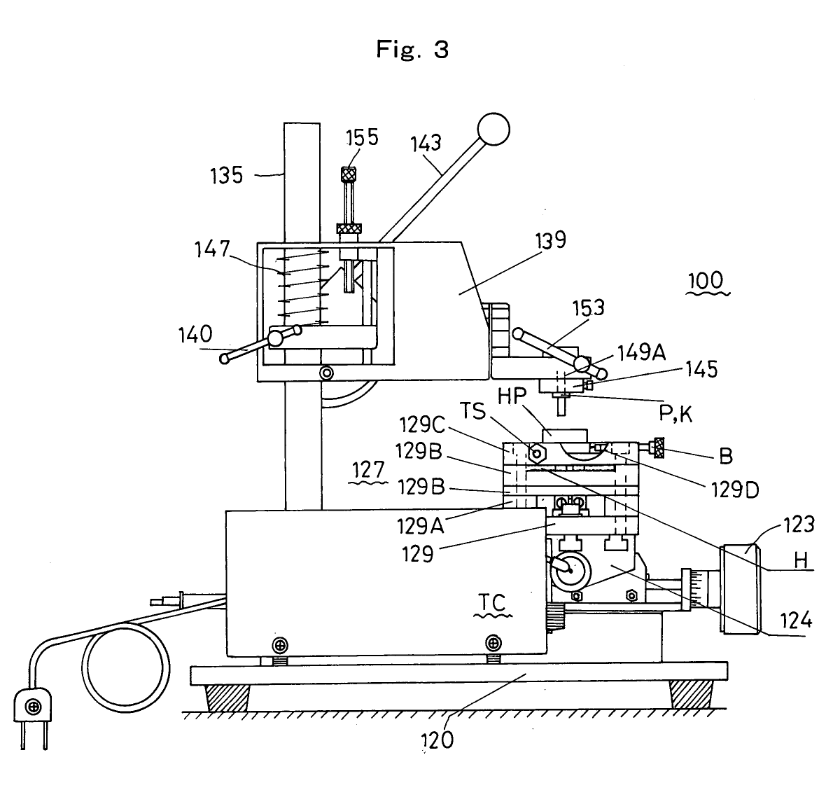

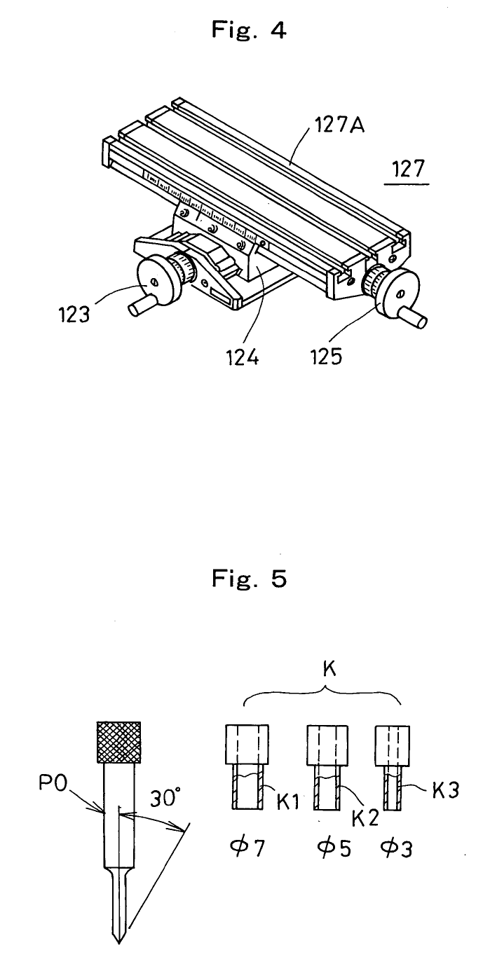

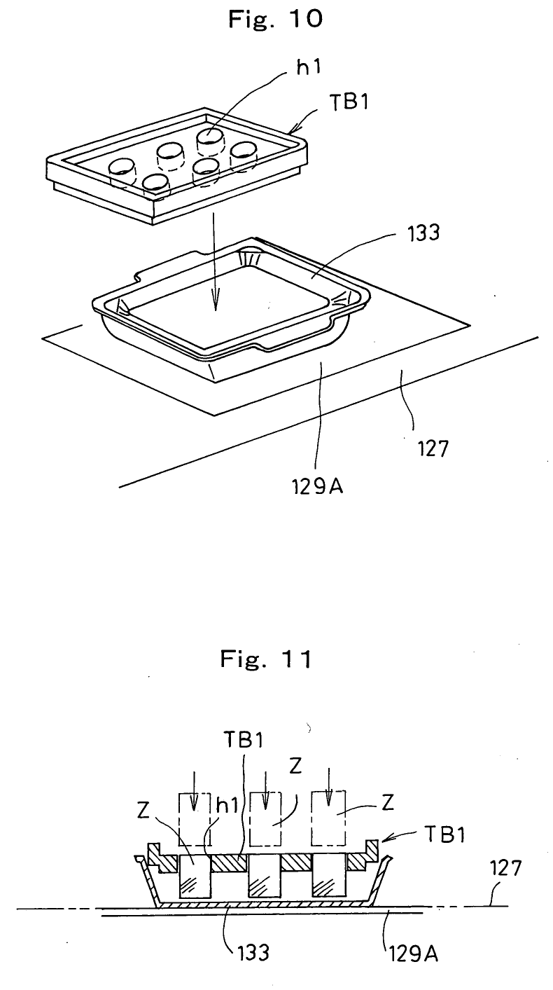

A first embodiment of the present invention, related to a method for constructing tissue blocks, and a tissue punching instrument, and tissue blocks used for that method, will now be described with reference to FIGS. 1 through 15.

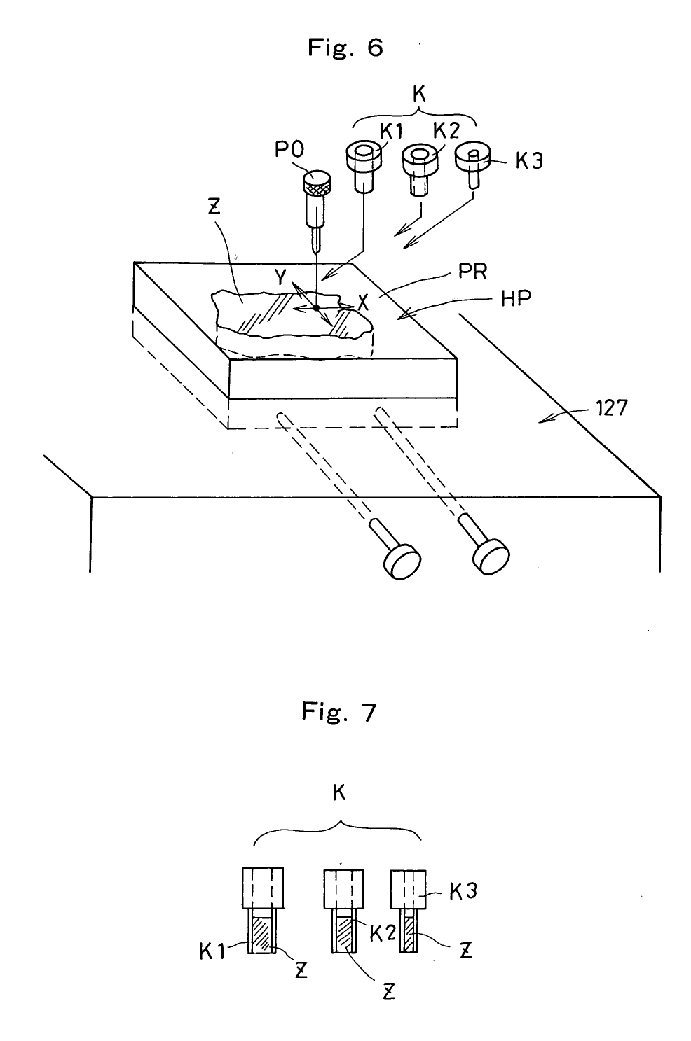

As illustrated in FIG. 6, a tissue punching instrument 100, according to the first embodiment of the present invention, comprises a stage 127 and tubular blades K. The stage 127 serves for accepting and fixing formalin fixed paraffin block HP of an organ Z, which has been obtained by surgical operation or biological test. Each of the tubular blades K is, for example, in a cylindrical shape, and serves for punching the tissue from the organ Z. The stage 127 is provided with a horizontal direction positioning mechanism, by which the stage 127 may be moved in X axis and Y axis directions, so that the front end of the tubular blade K may be positioned substantially at the target part of the organ Z. The horizontal direction positioning mechanism may be provided in the tubular blade K. According to the tissue punching instrument 100 of the present embodiment, as illustrated in FIGS. 1 through 5, a base 120 is provided with a holder 145, and a tissue punching lever 143 which moves the holder 145 in upward and downward directions. The holder 145 holds the tubular blade K and a positioning pin P0, which can be replaced alternatively. The formalin fixed paraffin block HP, containing the organ Z, is mounted on the stage 127, and then is fixed on a predetermined position. The target position, from which the tissue is obtained, may be determined by the positioning pin P0.

The positioning pin P0 is then replaced by the. tubular blade K. The tubular blade K is pressed in the downward direction, and inserted in the formalin fixed paraffin block HP, whereby the organ (tissue) Z is punched out of the formalin fixed paraffin block HP. The stage 127 is provided with a Y axis rotation knob 123 and an X axis rotation knob 125, by which the front end of the positioning pin P0 or the front end of the tubular blade K may be positioned relatively, from the respective original scale positions in X axis and Y axis directions, to the target part of the tissue, through movement of the stage 127. The stage 127 is also provided with a heater 129A for heating the formalin fixed paraffin block HP. The preferable heating temperature may be from 40° C. to 50° C., because the paraffin block HP may become softer, and can be punched without giving any damage thereto. Thus, it is possible to obtain perfect condition of organ tissues without having any damage. The heater 129A is not indispensable, and it is also possible to carry out the above punching operation at normal temperature.

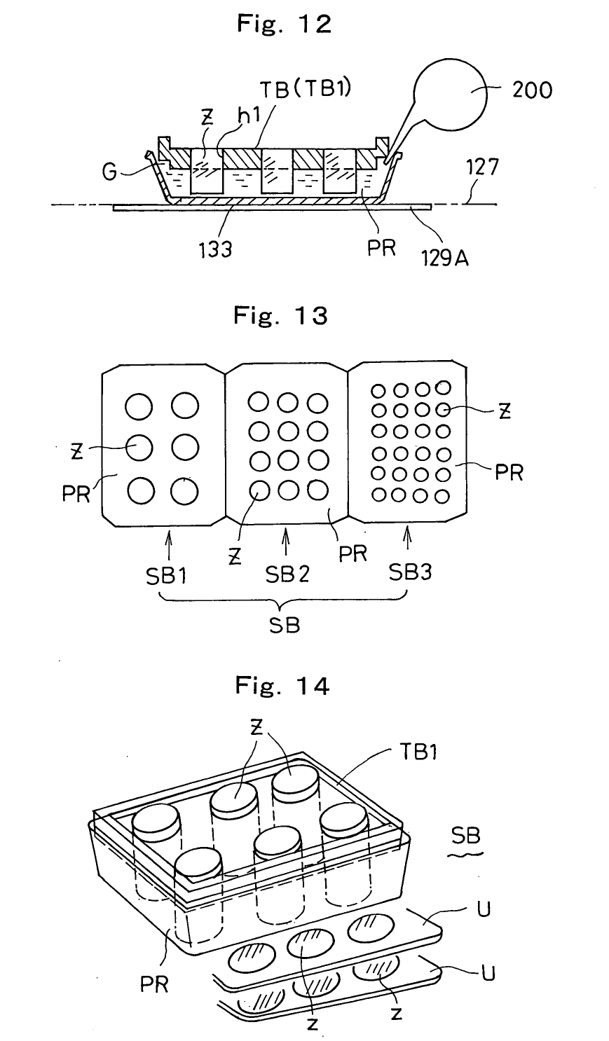

There are three types of the tubular blade K, at outer diameters of 3 mm, 5 mm and 7 mm, and the respective tubular blades K punch the organ tissues Z at the corresponding diameters. There are also three types of tissue blocks TB1, TB2 and TB3 prepared in advance, corresponding to the above three types of the organ tissue Z. The tissue block (tissue holder) is, for example, a rectangular plate made of synthetic resin, in which a plurality of holes h1, h2 and h3 has been formed. The inner diameters of the holes h1, h2 and h3 are, 3.5 mm, 5.5 mm and 7.5 mm, respectively, which are very close to those of the tubular blades K, i.e. 3.0 mm, 5.0 mm and 7.0 mm, respectively, and the organ tissues Z are planted in these holes h1, h2 and h3. As illustrated in FIG. 12, paraffin PR is filled from a gap G, and the organ tissues Z are inserted in the paraffin PR to be integrated with each other, whereby an array block SB is obtained.

The structure of the tissue punching instrument 100 will be discussed in detail, with reference to FIGS. 1 through 4. The tissue punching instrument 100 is provided with the horizontal direction positioning mechanism, comprising a Y axis table 124 and an X axis table 127A, on the base 120. The Y axis table 124 moves in the front and rear directions by the Y axis rotation knob 123, and the Y axis table 127A moves in the right and left directions by the X axis rotation knob 125. The stage 127 is provided on the horizontal direction positioning mechanism. There are the heater 129A, two intermediate layers 129B, and a holder plate 129C, respectively provided on the X axis table 127A. The holder plate 129C serves for mounting and fixing of the formalin fixed paraffin block HP of the organ Z thereon, and is provided with a heating body 129D in a recessed shape at the center thereof. The heating body 129D has two fastening screws B, respectively serving for engaging with and fixing the formalin fixed paraffin block HP. The temperature of the heating body 129D is detected by a thermal sensor TS, and a thermal controller TC controls the heater 129, so that the heating body 129D maintains the temperature from 40° C. to 50° C.

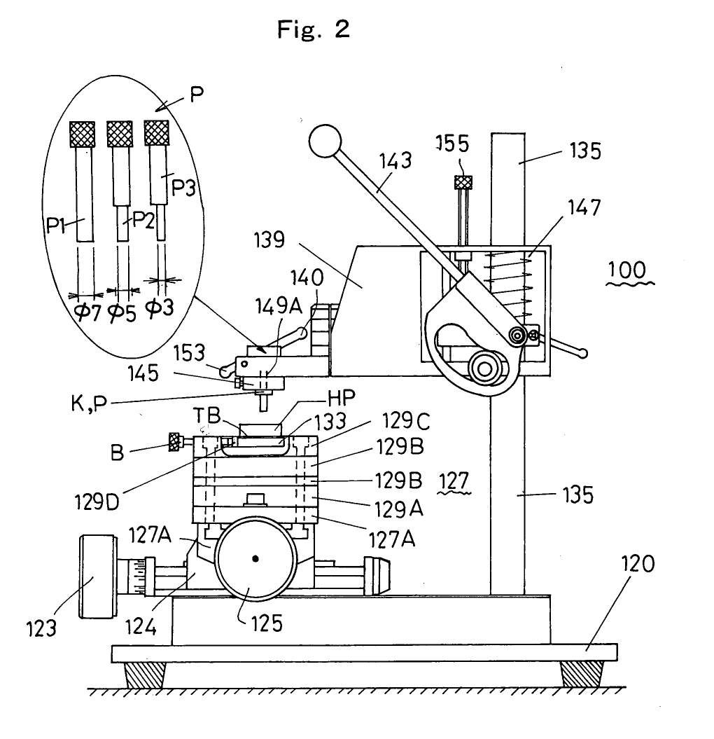

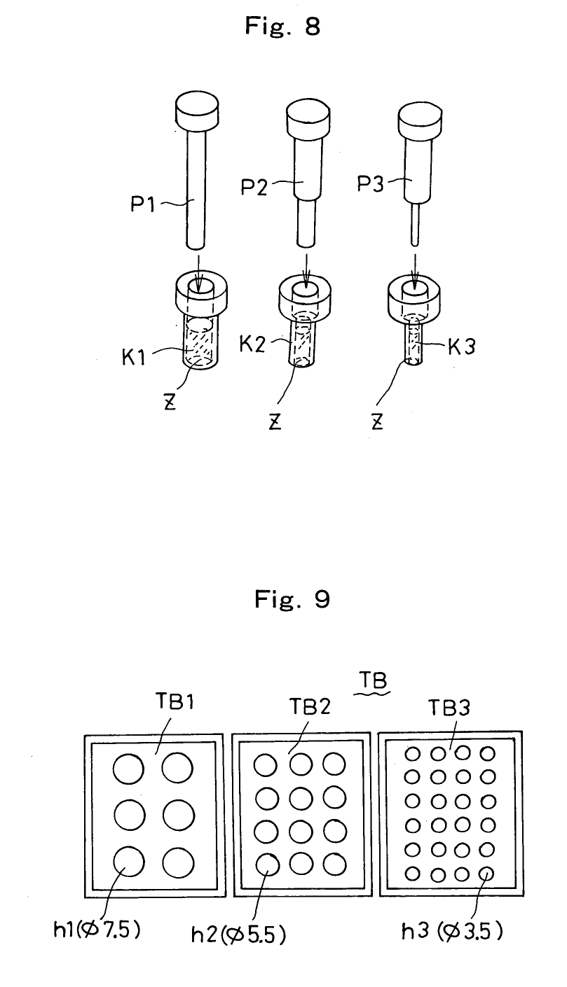

There is a pillar 135 in the rear of the base 120, and the head 139 is engaged with the upper portion of the pillar 135. The head 139 is movable in the upward and downward directions along the pillar 135, and may be stopped at any position by a head position fixing lever 140. The tissue punching lever 143 is provided on the right of the head 139, and the holder 145, holding the tubular blade K, is moved in the upward and downward directions by operation of the tissue punching lever 143. There is a coil spring 147 inside the head 139, applying force to the holder 145 in the upward direction, so that the holder 145 may be maintained at the upper position in a normal state. As illustrated in FIGS. 2 and 8, there are three types of tissue extracting rod P (P1, P2 and P3), corresponding to (somewhat smaller than) the respective outer diameters of the tubular blades K (3 mm, 5 mm and 7 mm). There is a stopper 155, by which the up-down stroke of the tubular blade K is controlled.

According to the above structure, when array blocks are constructed, the stage 127 is moved horizontally in X axis and Y axis directions from the respective original scale positions, whereby the front end of the tubular blade K is relatively positioned, substantially in the target part of the organ tissue Z, thus the organ tissue Z is punched by the tissue extracting rod P. The organ tissue Z punched by the tissue extracting rod P is then planted in the holes h1, h2 and h3 of the tissue block TB, fixed on an embedding pan 133. As illustrated in FIG. 9, there are three types of tissue blocks TB1, TB2 and TB3, respectively accepting, for example, 6 samples, 12 samples and 24 samples to be planted therein. Accordingly, as illustrated in FIG. 12, the liquid paraffin PR is filled from the gap G between the embedding pan 133 and the tissue block TB, or from grooves on the both sides of the tissue block TB, whereby the organ tissue Z and the liquid paraffin PR are integrated, and the liquid paraffin PR is solidified. Thereafter, the tissue block TB is removed out of the embedding pan 133, whereby the array blocks (array of tissues) SB (SB1, SB2 and SB3) are constructed.

As discussed above, the tissue punching instrument 100 is provided with the heater 129A so that the formalin fixed paraffin block HP is heated at temperature from 40° C. to 50° C. on the stage 127, and with the positioning pin P0 by which the tubular blade K is positioned accurately at the target of the organ Z. In addition, the heater 129A may also serve for heating the liquid paraffin PR filled in the gap G between the embedding pan 133 and the tissue block TB on the stage 127. Thus, the heater 129A on the stage 127 may heat both the formalin fixed paraffin block HP and the liquid paraffin PR. The positioning pin P0 is not indispensable, and the tubular blade K may serve as a pointer for positioning at a predetermined position on the organ tissue Z.

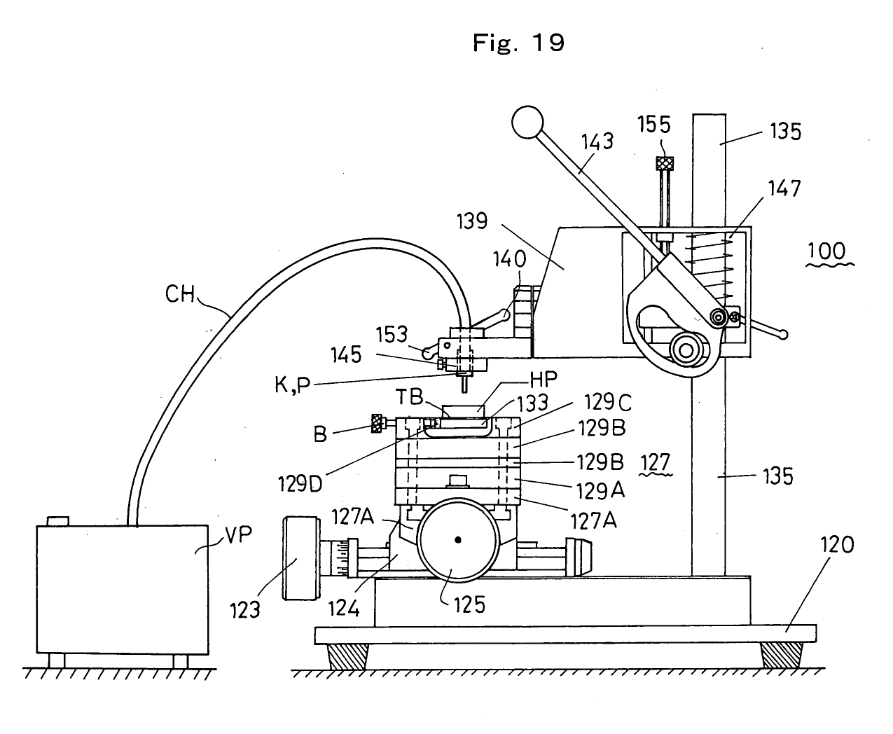

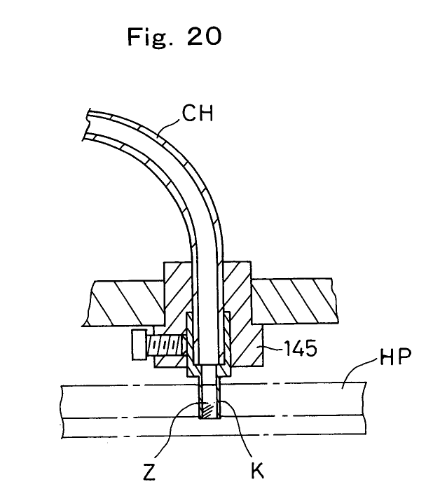

FIG. 19 and 20 illustrate a second embodiment of the present invention. As shown in FIGS. 19 and 20, according to the tissue punching instrument 100, the tubular blade K is provided with a vacuum suction apparatus. According to the tissue punching instrument 100 as discussed above, the tissue punching lever 143 is pushed in the downward direction, whereby the tubular blade K is pushed down into the formalin fixed paraffin block HP, so that the organ tissue Z is punched out of the formalin fixed paraffin block HP. However, the tissue and paraffin are not uniform, and there are several cases in which the appropriate tissue cannot be extracted. On the other hand, according to the present embodiment, the vacuum suction apparatus serves for the vacuum suction inside the tubular blade K. The vacuum suction apparatus is provided with a vacuum pump VP, and an end of a tube CH is connected to an air inlet of the vacuum pump VP. The other end of the tube CH is connected to the rear end of the tubular blade K via a hole of the holder 145, and when the vacuum pump VP is actuated, the vacuum suction is carried out inside the tubular blade K. With this structure, when the tissue punching lever 143 is pushed in the downward direction, whereby the tubular blade K is also pushed down in the downward direction into the formalin fixed paraffin block HP, in order to punch the organ tissue Z out of the formalin fixed paraffin block HP, if there is any difficulty in obtaining the organ tissue Z, the vacuum pump VP may solve this problem. When the vacuum pump VP is actuated, the negative pressure is given in the space inside the tubular blade K, whereby the organ tissue Z, which has been adhering to the formalin fixed paraffin block HP, can be pulled into the tubular blade K without fail. The vacuum suction apparatus may be used on any occasion, by considering the condition of tissue or paraffin.

A method for constructing array blocks according to the present invention will now be discussed, with reference to FIGS. 6 through 14, and a flow chart of FIG. 15. First, the formalin fixed paraffin block HP of the organ Z, which has been extracted by surgical operation of biological test, is prepared at step S1. The formalin fixed paraffin block HP is fixed on the stage 127, and heated at temperature from 40° C. to 50° C. by the heater 129A (step S2). When the formalin fixed paraffin block HP is sufficiently heated, the Y axis rotation knob 123 and the X axis rotation knob 125 are rotated from the original scale positions, whereby the stage 127 is moved, and the front end of the tubular blade K is relatively positioned, substantially at the target position (step S3). At that time, the accurate position of the target tissue is also determined by the positioning pin P0 (step S4). Thereafter, when the tissue punching lever 143 is pressed in the downward direction, the tubular blade K punches the tissue (step S5). There are three types of tubular blade K, of which respective outer diameters are 3 mm, 5 mm and 7 mm, and appropriate one may be selected among them according to the required size of the tissue. When there is any difficulty in extracting the organ tissue Z from the formalin fixed paraffin block HP, the vacuum pump VP may be actuated, so that the negative pressure is given in the space inside the tubular blade K, whereby the organ tissue Z, which has been adhering to the formalin fixed paraffin block HP, can be pulled into the tubular blade K without fail. Thereafter, the tissue extracting rod P, of which outer diameter is corresponding to (somewhat smaller than) the outer diameter of the tubular blade K, is used for extracting the tissue from the tubular blade K (step S6).

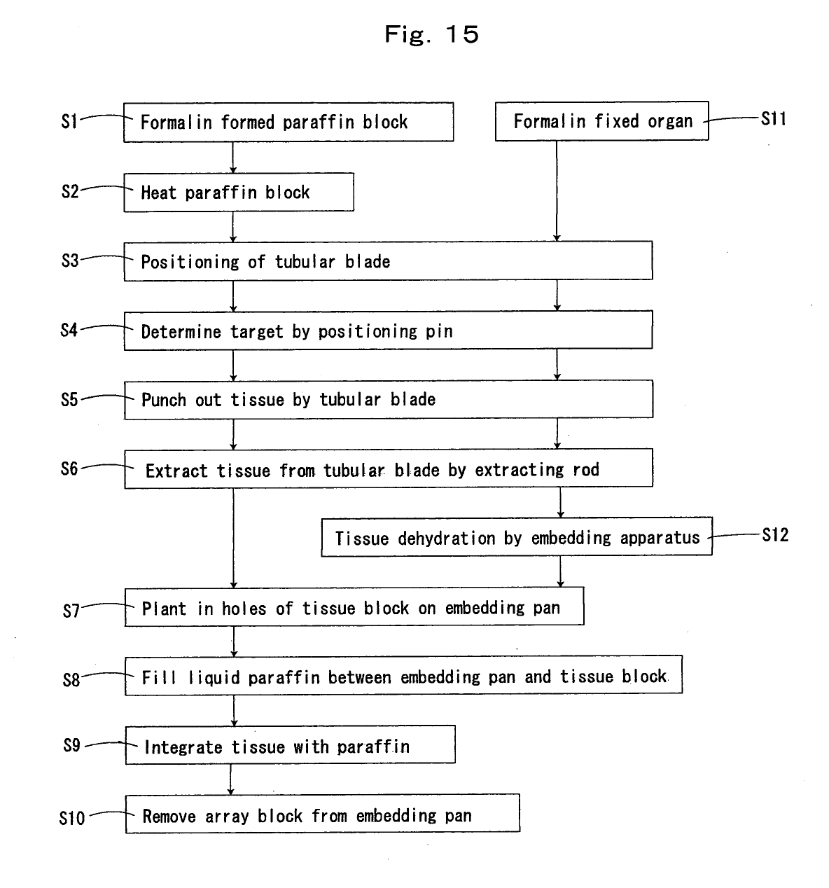

The obtained organs Z are then planted in the respective holes h of the tissue block TB, fixed on the embedding pan 133 (step S7). There are also three types of tissue block, corresponding to the extracted organ tissues Z having any diameters among 3 mm, 5 mm and 7 mm, so that any one tissue block may be used selectively. The respective tissue blocks TB accept, 24 samples (for the diameter of 3 mm), 12 samples (for the diameter of 5 mm) and 6 samples (for the diameter of 7 mm). Thereafter, the heated liquid paraffin PR is filled, from the gap G between the embedding pan 133 and the tissue block TB, or from the grooves on the both sides of the tissue block TB (step S8). The liquid paraffin PR is filled from a paraffin supplier 200, and left for a while on the heater 129A of the stage 127, so that the organ Z and the liquid paraffin PR may be integrated (step S9). It is also possible to carry out heating and cooling on a known heater built-in type of embedding apparatus mounted on the stage 127, instead of using the heater 129A of the stage 127. Thereafter, the integrated organs Z and the tissue blocks TB are moved to the cool stage 127, and when the sufficient cooling is done, the prepared array block SB is removed from the embedding pan 133 (step S10). According to the steps as discussed above, it is possible to obtain the array block SB as illustrated in FIG. 13. As illustrated in FIG. 14, the thus obtained array block SB is sliced by microtome likewise the case of ordinary paraffin block, whereby sections U may be obtained. With reference to the array block SB according to the present invention, the organs Z are held by the tissue block TB, and are also integrated with the paraffin PR. Thus, there are thin organ tissues z securely held inside the sliced section U.

According to the method of constructing array blocks of the present invention, it is also possible to obtain the array block SB, by directly punching tissues out of a formalin fixed organ itself. In this case, the formalin fixed organ Z is prepared at step 11. This organ Z is fixed on the stage 127, and is punched by the tubular blade K (steps S3-S6). The size may be selected among 3 mm, 5 mm and 7 mm. The punched organ tissues Z are then dehydrated and defatted by known tissue embedding apparatus (step S12). The dehydration and defatting are not indispensable, and it is also possible to use the punched organs Z without those treatments. Thereafter, likewise the above steps, the organ tissues Z are planted in the holes h of the tissue block TB, the liquid paraffin PR is filled, and the integration and the cooling solidification are carried out (steps S7-S10). Accordingly, the array block SB, selected among 24 samples, 12 samples and 6 samples, is prepared. The prepared array block SB is then sliced by microtome.

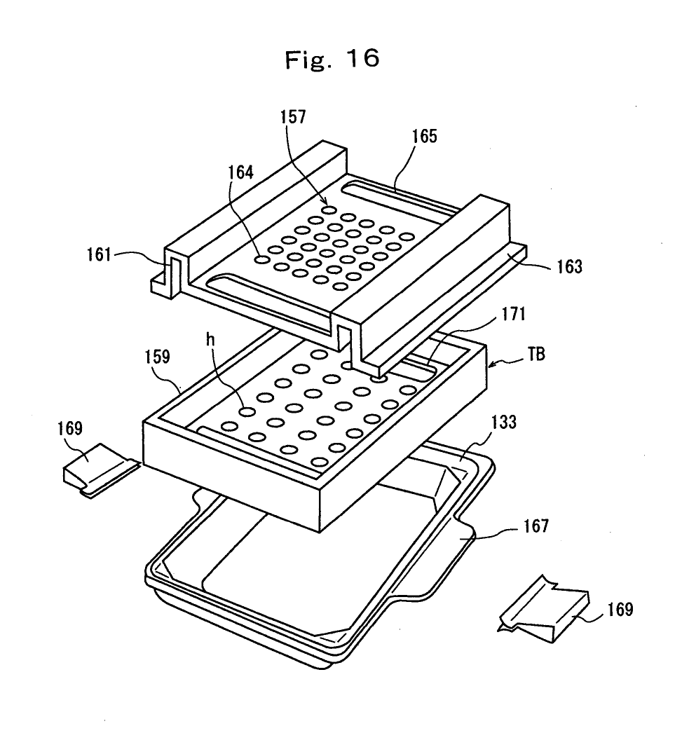

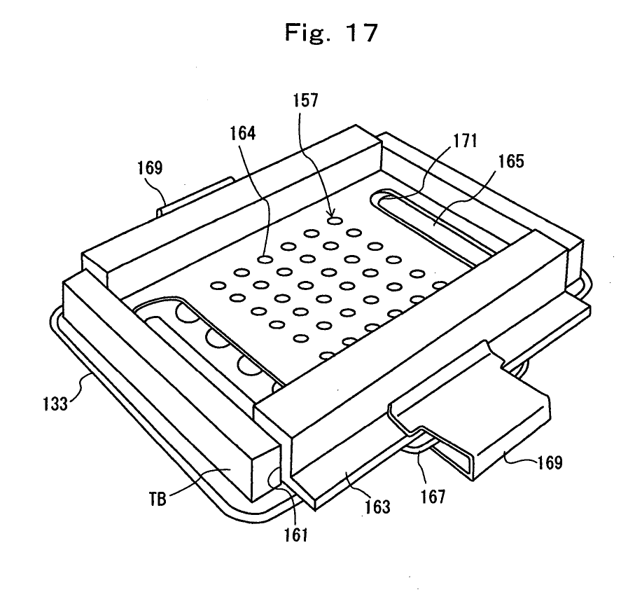

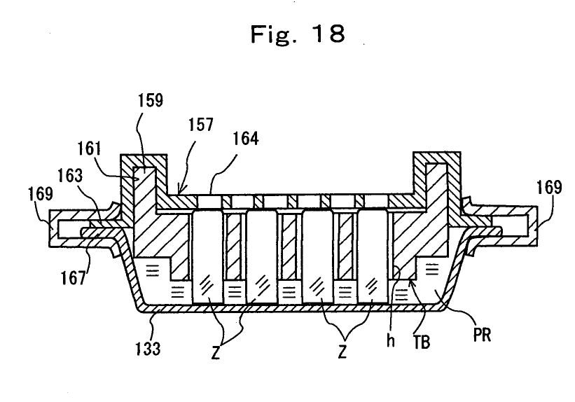

With reference to the array block SB constructed by directly punching tissues out of the formalin fixed organ itself, as illustrated in FIGS. 16 through 18, when the punched organ tissues Z are dehydrated and defatted, and when the liquid paraffin PR is filled for integration, it is also possible to use another type of tissue block TB, provided with an upper cover 157 for preventing movement of the organ tissues Z. The rectangular shape of the upper cover 157 is substantially in the same size as that of the tissue block TB, having a pair of engagement grooves 161 in the elongating direction, which are engaged with a pair of engagement walls 159 correspondingly provided on the both sides of the tissue block TB. The each of the engagement grooves 161 has an engagement part 163 extending outwardly. There are may holes 164 on a panel surface of the upper cover 157 serving for smooth input of the liquid paraffin PR. Further, there are slits 165, respectively provided along the shorter sides of the panel surface of the upper cover 157, also serving for smooth input of the liquid paraffin PR. There are also slits 171 on the respective shorter sides of the tissue block TB, at the positions corresponding to the slits 165 of the upper cover 157.

With reference to the constructing of the array block SB by using the tissue block TB provided with the upper cover 157, first, the organ tissues Z, which have been punched directly out of the formalin fixed organ itself, are planted in the holes h of the tissue block TB fixed on the embedding pan 133. Thereafter, the upper cover 157 is placed on the tissue block TB, so that the panel surface of the upper cover 157 may press the organ tissues Z. The engagement walls 159 are inserted in and engaged with the engagement grooves 161, and the engagement parts 163 of the upper cover 157 are placed on the corresponding engagement parts 167, respectively formed on the upper rims on the both sides of the embedding pan 133 in the elongating direction. The each of the engagement parts 163 of the upper cover 157, and each of the corresponding engagement parts 167 of the embedding pan 133, are banded by clip 169. Accordingly, the upper cover 157 is fixed on the tissue block TB, and at the same time, the upper cover 157 and the tissue block TB are also fixed on the embedding pan 133, whereby the movement of the organs Z. held inside the holes h, are prohibited. Therefore, it is possible to directly apply dehydration and defatting smoothly by alcohol and xylene in an automatic embedding apparatus, and continuously, it is also possible to directly fill the heated liquid paraffin PR smoothly, from the slits 165, 171 on the both sides of the upper cover 157 as well as on the both sides of the tissue block TB, and also from the many holes 164 on the panel surface of the upper cover 157. The organ tissues Z are held in the holes h, and the movement of the tissues Z is prohibited by the upper cover 157. Therefore, if there is any vibration during filling of the liquid paraffin PR, it is possible to prohibit the movement of the tissues Z, whereby the high quality array blocks SB, in which the each organ tissue Z has been positioned accurately, can be constructed easily without fail.

The present invention has the following merits. First, according to the method for constructing array blocks, the liquid paraffin is filled after inserting the tissues in the newly provided tissue block. Thus, it is possible to integrate the tissues and paraffin smoothly, whereby neat and stable array blocks may be constructed. Further, the array blocks may also be constructed by directly punching the tissues out of the fixed organ itself, thus the applicable scope becomes wider. When the sliced array block is analyzed, it is possible to use an ordinary slide glass, and any special slide glass, adhesive tape or UV irradiation is no longer required.

According to the method for constructing array blocks of the present invention, the formalin fixed paraffin block HP is heated at temperature from 40° C. to 50° C. by the heater 129A of the stage 127. Therefore, the softened paraffin block HP can be punched, and there is almost no damage to the paraffin block HP, whereby the perfect condition of organ tissues without having any damage may be obtained. When the tissue Z is punched by the tubular blade K, if the vacuum suction is done inside the tubular blade K, the tissue Z can be extracted without fail.

According to the tissue punching instrument 100 of the present invention, the outer diameters of the tubular blades K are from 3 mm to 7 mm. Thus, it is possible to obtain relatively wide range of organ tissues Z, which can be applicable to ordinary tissue diagnosis. Further, the obtained tissues can also be used sufficiently for diagnosis of special staining and immunostaining, fluorescence in situ hybridization (FISH method), and other research purposes.

Further, according to the tissue punching instrument 100 of the present invention, the positioning pin P0 is provided for accurately positioning the tubular blade K at the target tissue. Thus, it is possible to carry out the positioning of the target tissue accurately, whereby the tissue punching operation may also become very simplified as compared with conventional operation, and even a fresh operator may handle the instrument easily within a short period of time. Therefore, the total cost for operation may be reduced, and applicable scope may extend to ordinary pathological purposes. When the tubular blade K of the tissue punching instrument 100 is provided with the vacuum suction apparatus, it is possible to extract the organ tissue Z without fail.

The operation of the tissue punching instrument 100 according to the present invention has been greatly simplified, thus it is possible to handle, even for a fresh operator, to handle the instrument easily within a short period of time.

According to the array block SB of the present invention, many samples may be placed on a single slide glass, and the diagnosis can be carried out. Therefore, from the viewpoints of ordinary pathological purposes and research purposes, as compared with conventional array for diagnosis, using only a single section of a single organ, the total cost for diagnosis may be reduced to almost tithe or less.

The three types of array blocks SB respectively have 24, 12 and 6 samples planted therein. Thus, it is possible to carry out diagnosis of 24, 12 or 6 samples at the same time of single staining.

The array block SB according to the present invention may also be used for any immunostaining in which special stain or expensive antibody is required other than ordinary hematoxylin-eosin (HE) stain, thus it is possible to reduce the cost remarkably.

Further, the array block SB according to the present invention is also applicable to analysis of chromosome and gene of cancer, etc., using fluorescence in situ hybridization (FISH method), at remarkably reduced research cost.

When various different organs are planted in the array block SB of the present invention, and stained by special antibody or probe, it is possible to use as control slide.

Further, with reference to the array block SB of the present invention for pathological purposes, after roughly trimming a target part of organ from the macroscopic viewpoint, it is possible to punch a plurality of samples at the same time by using the tissue punching instrument according to the present invention. Therefore, lesion parts of the various organs can be checked by a few slide glasses, and as compared with conventional instruments, in which one section of one organ requires one slide glass, it is possible to reduce the cost for slide glasses, cover glasses and reagents.

According to the tissue block TB of the present invention, the punched tissues are arranged in the holes, and the top side of each tissue is facing protrusively toward the embedding pan. The liquid paraffin is filled from the gap between the embedding pan and the tissue block, and then solidified. Accordingly, it is possible to construct the array block SB easily and efficiently, in which the array of tissues and the paraffin have been integrated.

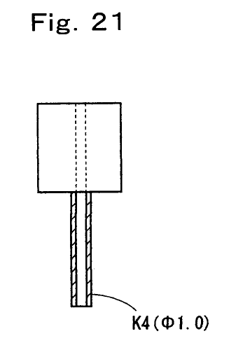

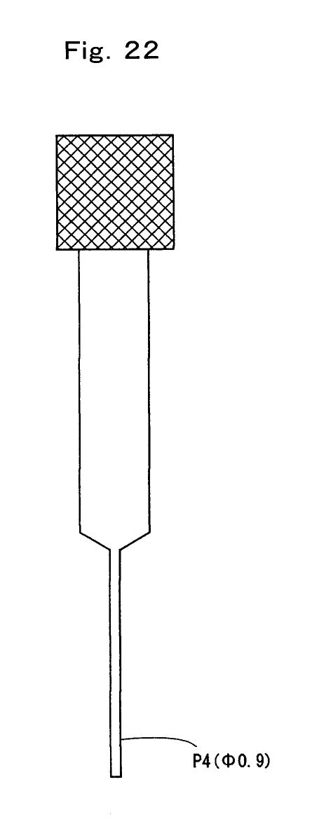

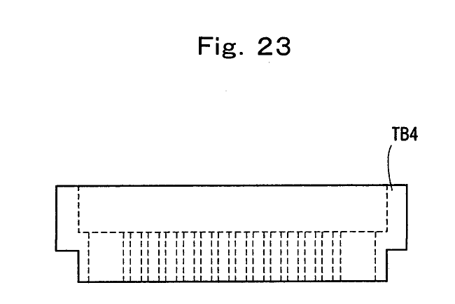

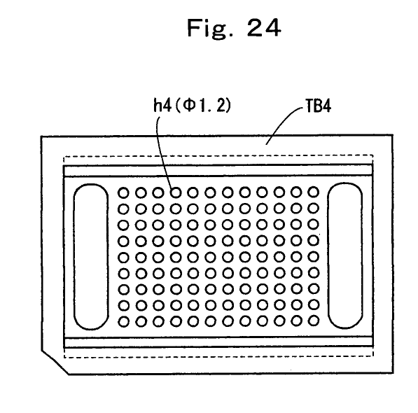

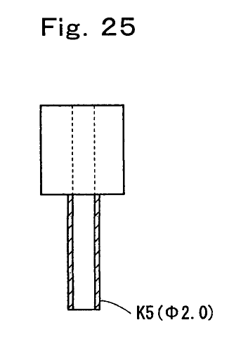

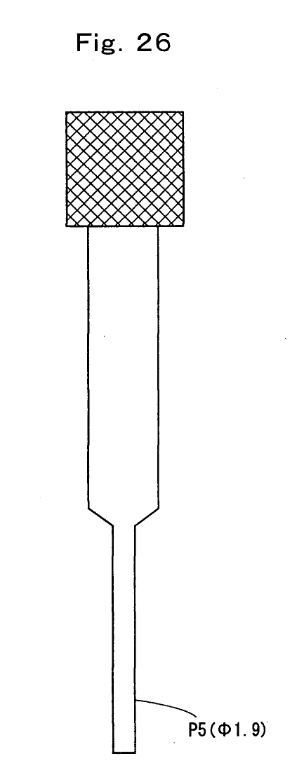

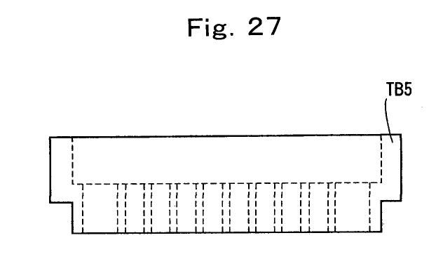

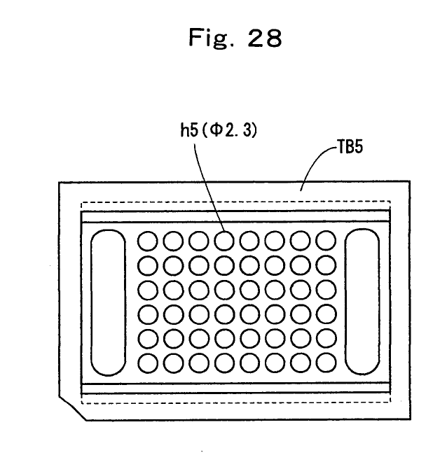

The present invention is not limited to the embodiments as discussed above, and any modification may be done without departing the spirit of the present invention. In particular, with reference to the tissue punching instrument, the outer diameter and shape of the tubular blade, and the inner diameter, shape and number of the corresponding holes of the tissue block may be determined arbitrarily. For example, the outer diameter of the tubular blade K may be reduced, such as 1 mm or 2 mm, and the corresponding sizes of tissue extracting rods P and the tissue block TB may be arranged. As illustrated in FIGS. 21 through 24, when the outer diameter of a tubular blade K4 is 1 mm, the outer diameter of a tissue extracting rod P4 is 0.9 mm, and each inner diameter of holes h4 of a tissue block TB4 is 1.2 mm. There are 108 holes h4 in the tissue block TB4. On the other hand, as illustrated in FIGS. 25 through 28, when the outer diameter of a tubular blade K5 is 2 mm, the outer diameter of a tissue extracting rod P5 is 1.9 mm, and each inner diameter of holes h5 of a tissue block TB5 is 2.3 mm. There are 48 holes h5 in the tissue block TB5. There may also be other examples likewise the above structure, and although the explanation is not made here, these other examples are of course included in the scope of the present invention. Further, it is not always necessary to provide the heater for heating paraffin, and even in the case of providing the heater, the heating temperature may be determined arbitrarily. The X axis rotation knob and the Y axis rotation knob, for moving the stage, may be controlled manually or automatically. The details of each structural member may be altered freely, as long as the essential structure of the present invention is maintained. Further, the method for constructing array blocks may be altered, as long as the essential method of the present is maintained.

1. A method for constructing array blocks, comprising steps of:
fixing a formalin fixed paraffin block of an organ on a stage;
moving said stage and relatively positioning a front end of a tubular blade at a target part of said organ;
punching tissue of said organ by said tubular blade;
extracting said tissue from said tubular blade by a tissue extracting rod;
preparing a tissue block in advance having a plurality of holes, each of said holes having inner diameter very close to the outer diameter of said tubular blade;
planting extracted tissues in said holes of said tissue block placed on an embedding pan, so that the front end of said tissue is protrusively facing toward said embedding pan; and
filling liquid paraffin into a gap between said embedding pan and said tissue block, and solidifying said liquid paraffin, whereby said tissue and paraffin are integrated.

2. A method for constructing array blocks, comprising steps of:
fixing an organ on a stage after formalin fixing;
moving said stage and relatively positioning a front end of a tubular blade at a target part of said organ;
punching tissue of said organ by said tubular blade;
extracting said tissue from said tubular blade by a tissue extracting rod;
preparing a tissue block in advance having a plurality of holes, each of said holes having inner diameter very close to the outer diameter of said tubular blade;
planting extracted tissues in said holes of said tissue block fixed on an embedding pan, so that the front end of said tissue is protrusively facing toward said embedding pan; and
filling liquid paraffin into a gap between said embedding pan and said tissue block, and solidifying said liquid paraffin, whereby said tissue and paraffin are integrated.

3. The method for constructing array blocks as claimed in claim 1, characterized in that said formalin fixed paraffin block is heated at temperature from 40° C. to 50° C. by a heater of said stage.

4. The method for constructing array blocks as claimed in claim 1, further characterized in that said liquid paraffin, filled into said gap between said embedding pan and said tissue block, is heated by a heater, whereby said tissue and paraffin are integrated.

5. The method for constructing array blocks as claimed in claim 2, further characterized in that said liquid paraffin, filled into said gap between said embedding pan and said tissue block, is heated by a heater, whereby said tissue and paraffin are integrated.

6. The method for constructing array blocks as claimed in claim 1, further characterized in that the outer diameter of said tubular blade is from 3 mm to 7 mm.

7. The method for constructing array blocks as claimed in claim 2, further characterized in that the outer diameter of said tubular blade is from 3 mm to 7 mm.

8. The method for constructing array blocks as claimed in claim 3, further characterized in that the outer diameter of said tubular blade is from 3 mm to 7 mm.

9. The method for constructing array blocks as claimed in claim 4, further characterized in that the outer diameter of said tubular blade is from 3 mm to 7 mm.

10. The method for constructing array blocks as claimed in claim 5, further characterized in that the outer diameter of said tubular blade is from 3 mm to 7 mm.

11. The method for constructing array blocks as claimed in claim 1, further characterized in that vacuum suction is done inside said tubular blade during punching of tissue by said tubular blade.

12. The method for constructing array blocks as claimed in claim 2, further characterized in that vacuum suction is done inside said tubular blade during punching of tissue by said tubular blade.

13. The method for constructing array blocks as claimed in claim 3, further characterized in that vacuum suction is done inside said tubular blade during punching of tissue by said tubular blade.

14. The method for constructing array blocks as claimed in claim 4, further characterized in that vacuum suction is done inside said tubular blade during punching of tissue by said tubular blade.

15. The method for constructing array blocks as claimed in claim 5, further characterized in that vacuum suction is done inside said tubular blade during punching of tissue by said tubular blade.

16. The method for constructing array blocks as claimed in claim 6, further characterized in that vacuum suction is done inside said tubular blade during punching of tissue by said tubular blade.

17. The method for constructing array blocks as claimed in claim 7, further characterized in that vacuum suction is done inside said tubular blade during punching of tissue by said tubular blade.

18. The method for constructing array blocks as claimed in claim 8, further characterized in that vacuum suction is done inside said tubular blade during punching of tissue by said tubular blade.

19. The method for constructing array blocks as claimed in claim 9, further characterized in that vacuum suction is done inside said tubular blade during punching of tissue by said tubular blade.

20. The method for constructing array blocks as claimed in claim 10, further characterized in that vacuum suction is done inside said tubular blade during punching of tissue by said tubular blade.

21. A tissue punching instrument comprising:
a base;
a tubular blade mounted on said base for punching tissue by an up-down operation mechanism; and
a stage mounted on said base, for fixing a formalin fixed paraffin block, and for relatively positioning a front end of said tubular blade substantially at a target part of an organ through movement of said stage in X axis and Y axis directions by a horizontal positioning mechanism.

22. The tissue punching instrument as claimed in claim 21, further comprising a heater for heating said formalin fixed paraffin block.

23. The tissue punching instrument as claimed in claim 21, further characterized in that the outer diameter of said tubular blade is from 3 mm to 7 mm.

24. The tissue punching instrument as claimed in claim 22, further characterized in that the outer diameter of said tubular blade is from 3 mm to 7 mm.

25. The tissue punching instrument as claimed in claim 21, further comprising a positioning pin provided on said base, selectively used in place of said tubular blade, for determining an accurate position of tissue at said target part.

26. The tissue punching instrument as claimed in claim 22, further comprising a positioning pin provided on said base, selectively used in place of said tubular blade, for determining an accurate position of tissue at said target part.

27. The tissue punching instrument as claimed in claim 23, further comprising a positioning pin provided on said base, selectively used in place of said tubular blade, for determining an accurate position of tissue at said target part.

28. The tissue punching instrument as claimed in claim 24, further comprising a positioning pin provided on said base, selectively used in place of said tubular blade, for determining an accurate position of tissue at said target part.

29. The tissue punching instrument as claimed in claim 21, further comprising a vacuum suction apparatus inside said tubular blade.

30. The tissue punching instrument as claimed in claim 22, further comprising a vacuum suction apparatus inside said tubular blade.

31. The tissue punching instrument as claimed in claim 23, further comprising a vacuum suction apparatus inside said tubular blade.

32. The tissue punching instrument as claimed in claim 24, further comprising a vacuum suction apparatus inside said tubular blade.

33. The tissue punching instrument as claimed in claim 25, further comprising a vacuum suction apparatus inside said tubular blade.

34. The tissue punching instrument as claimed in claim 26, further comprising a vacuum suction apparatus inside said tubular blade.

35. The tissue punching instrument as claimed in claim 27, further comprising a vacuum suction apparatus inside said tubular blade.

36. The tissue punching instrument as claimed in claim 28, further comprising a vacuum suction apparatus inside said tubular blade.

37. A tissue block, being able to be arranged on an embedding pan, characterized in that a plurality of holes has been formed, each of which inner diameter very close to the outer diameter of said tubular blade of said tissue punching instrument according to claim 21.
